(19) United States
(12) Patent Application Publication
(10) Pub. No.: US 2008/0014273 A1
(43) Pub. Date: Jan. 17, 2008

(54) Equine whole food supplements

(76) Inventor: Cynthia Montague, Charlottesville, VA (US)

Correspondence Address: MCGUIREWOODS, LLP, 1750 TYSONS BLVD, SUITE 1800, MCLEAN, VA, 22102, US

(21) Appl. No.: 11/826,432

(22) Filed: Jul. 16, 2007

Related U.S. Application Data
(60) Provisional application No. 60/830,674, filed on Jul. 14, 2006.

(51) Int. Cl. A61K 9/00; A61K 35/64; A61K 36/00; A61P 17/02; A61P 3/02; A61P 1/14; A61K 36/06

(52) U.S. Cl. 424/484; 424/539; 424/93.51

(57) ABSTRACT

Whole food equine formulations provided by the invention include well balanced nutritional supplements providing nutrients and vitamins, among other benefits. The whole food formulations generally promote, for example, better cell utilization instead of cell saturation, which results in increased overall general physiological and psychological health of subject animals including horses.

### CROSS REFERENCE TO RELATED APPLICATIONS

This application claims benefit of U.S. Provisional Application No. 60/830,674, filed Jul. 14, 2006, the disclosure of which is incorporated by reference herein in its entirety.

### BACKGROUND OF THE INVENTION

1. Field of the Invention

The invention is directed generally to equine food matrix and process for manufacturing.

2. Related Art

Modern society has forged industries to mass produce foods. However, the side effects of mass production of foods very often led to poor nutritional quality.

Similar problems or deficiencies exist in the production of equine food products where the manufacturing process of grains and feed typically require high heat milling, often over 400° F. Nutrients are destroyed at much lower temperatures. For example, several important and essential amino acids are destroyed at temperatures as low as 175° F.

Other potential or actual problems or deficiencies associated with animal feed include use of non-organic grains which may have been sprayed with pesticides and herbicides. Also, the grain may have been grown using genetically modified organisms (GMO), or genetically engineered (GE) organisms, perhaps in soil impregnated with chemical fertilizers, which still has perceived doubts of safety.

Typically, processed feed formulas of non-organic grains are then “fortified” to provide basic nutrition. Additives such as molasses may also be added in an attempt to cover a multitude of mass production side effects or deficiencies. Most tack shops and horse catalogs are filled with supplemental formulas which are purported to be the best supplement to feed one's horse. Often one must hope these supplements work for whatever ails a horse or for treating nutritional deficiencies.

The advent of multi-vitamin/mineral supplementation for horses mirrors the human supplements market including products of isolated fractionized nutrients made in a laboratory. These products are often formulated with mega potencies and called “natural” because label claims typically are required to provide only ten percent “natural” ingredients.

Investment costs are substantial in the horse industry. From breeding goals and horse show competitions to horse racing, overall health and maintenance costs are of significant concern. Moreover, the general physiological and psychological health of a horse in competition or show is of major concern to trainers, riders and owners alike. A calm, cooperative, vigorously healthy horse is highly desirable. Improved nutrition tends to lead to better performance and less ailments overall.

Recent human research has shown that nutrients need protein chaperones for delivery and that cells actually send out “requests” alerting protein chaperones to deliver nutrients to cells in need. Human research has shown that nutrients in whole foods, taken in natural form and not as fractionalized nutrients manufactured in a laboratory, has a higher cellular utilization because at least in part due to the presence of protein chaperones. Accordingly, there is a need for improved equine food supplements that can provide better nutrition and increased overall general physiological and psychological health for equine animals.

### SUMMARY OF THE INVENTION

The invention provides a process to manufacture an equine-edible matrix including whole food supplements, and methods of treatment for various equine health conditions or goals to alleviate short comings in supplements described above and not addressed by the prior art.

In one aspect, a solid equine-edible matrix having a discrete shaped form for the delivery of nutrients wherein said solid equine-edible matrix provides nutrients suitable for an equine diet that comprises a compressed mixture of a grain product and nutrients, said matrix having a maximum moisture content of less than 5% by weight, said matrix having been produced by the process of mixing said grain products with said nutrients; and thereafter removing moisture to a level at or below said maximum moisture content by exposure of said shaped object to a prolonged exposure at a temperature not higher than about 145° F., whereby moisture is removed from said shaped object resulting in said solid matrix having a discrete shaped form, whereby said solid matrix having a discrete shaped form contains an enhanced level of nutrients.

In another aspect, a method of equine treatment for treating digestion and absorption, comprising the step of administering an equine supplement over time, the supplement comprising the following ingredients: sprouted Quinoa, organic oat bran, sprouted mung beans, sprouted sunflower, papaya and honey.

In another aspect, a method of equine treatment for increasing circulation and providing a substrate for nitric oxide production is provided comprising the step of administering an equine supplement over time, the supplement comprising the following ingredients: dried ginger powder, sprouted Quinoa, sprouted Kamut wheat, oat bran, wheat germ, sprouted pumpkin seed, sprouted sesame seed, bananas, oranges, lemon, honey, Ginger extract, and yeast.

In another aspect, a method of equine treatment for supporting connective tissue healing and repair is provided comprising the step of administering an equine supplement over time, the supplement comprising the following ingredients: oat bran, wheat germ, sprouted millet, parsley, garlic powder, wheatgrass, apples, banana, red or Concord grapes, oranges and sprouted flax.

In yet another aspect, a method of equine treatment for reducing physical and mental stress is provided comprising the step of administering an equine supplement over time, the supplement comprising the following ingredients: sprouted buckwheat, sprouted mung dal, sprouted pumpkin, oat bran, Sensorile, apples, orange, bananas and honey.

In yet another aspect, a solid equine-edible matrix having a discrete shaped form for the delivery of nutrients wherein said solid equine-edible matrix provides nutrients suitable for an equine diet that comprises a compressed mixture of at least one grain product including at least one of oat bran and wheat germ and one or more nutrients including honey, the matrix having a maximum moisture content of less than 5% by weight, the matrix having been produced by the process of mixing said grain products with said nutrients; and thereafter removing moisture to a level at or below said maximum moisture content by exposure of said shaped object to a prolonged exposure at a temperature sufficient to achieve the maximum moisture content without destructive effects to said nutrients, whereby moisture is removed from said shaped object resulting in the solid matrix having a discrete shaped form, whereby said solid matrix having a discrete shaped form contains an enhanced level of nutrients.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

The embodiments of the invention and the various features and advantageous details thereof are explained more fully with reference to the non-limiting embodiments and examples that are described in the following description. Descriptions of well-known components or processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. The term “Equine” used herein includes all members of the equine family, such as horses, donkeys and zebras. Examples and descriptions presented herein typically refer to horses, but should be understood to include all members of the equine family.

It is understood that the invention is not limited to the particular methodology, protocols, devices, apparatus, materials, applications, etc., described herein, as these may vary. It is also to be understood that the terminology used herein is used for the purpose of describing particular embodiments only, and is not intended to limit the scope of the invention. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the invention.

The invention, in various embodiments described herein, generally provides whole food equine supplements or formulations to promote better overall equine health and processes for making the supplements. Also, methods for treating subject equine animals with the formulations are provided by the invention.

The formulations provide well balanced nutritional supplements including nutrients and vitamins, among other benefits as described herein. In one aspect, the whole food supplements promote better cell utilization instead of cell saturation, which results in increased overall general physiological and psychological health. Various embodiments also provide other or additional benefits as described herein.

Horses are members of the equine family Equidae and are at the center of attention of an ongoing major industry, namely the horse industry. Healthy horses are a basic goal for achieving quality blood lines and achieving reliable performance, whether in the show ring or on the track.

The whole food equine supplements provided by the invention include, in varying combinations, human grade herbals extracts, human grade nutracceuticals, sprouted organic seeds and grains, which typically are not genetically engineered (GE) and do not use genetically modified organisms (GMO). However, GE or GMO treated ingredients may be used, but is not preferred. The combinations provide improved nutrient delivery and, in select combinations, can be targeted to promote specific responses in horses, or to provide a specific treatment for an ailment and/or provide defense against acquiring an ailment.

Table 1 lists various individual ingredients from which specific ingredients may be selected to form the various whole food horse supplements, as described herein. The list of Table 1 is not meant to be exhaustive, but provides a summation of preferred ingredients (or nutrients) from which various formulations may be selectively based. Other ingredients (or nutrients) may be described elsewhere herein. The Table 1 ingredients and related exemplary characteristic or function(s) may be used in different combinations for specific targeted purposes and include:

TABLE 1

| INGREDIENT | CONTRIBUTION(S) AND/OR FUNCTIONAL CHARACTERISTIC(S) |
|---|---|
| Oat Bran | Contains plant lignans and beta glucans. Soluble Fiber and also |
| | helps hold whole food bars together |
| Wheat Germ | Naturally occurring Vitamin E and anti-oxidants |
| Millet | An alkaline grain, high in magnesium and tryptophan |
| Parsley Flakes | Rich in chlorophyll and may provide aid as a blood purifier. Dry. |
| Garlic | Helps synthesis of sulfur containing amino acids. Dry. |
| Wheat Grass | Rich in chlorophyll |
| Flax | High in essential fatty acids, particularly Omega 3 (preferred |
| | organic and sprouted) |
| Amaranth | High amounts of sulfur containing amino acids: Methionine, |
| | cysteine, and lysine (preferred organic sprouted) |
| Brewers Yeast | High in B vitamin complex |
| Quinoa | High in magnesium, manganese and iron |
| Kamut | High in lipids and a low glycemic index |
| Sesame | High in amino acid arginine —contributes essential amino acid |
| | Lysine (generally important for equines) |
| Cayenne | Vasodilator |
| Wheat Bran | Insoluble fiber. Dry. |
| Purple Kculi Corn | Anti-oxidant. |
| Mung Beans | Source of soluble and insoluble fiber, Vitamin K (preferred organic |
| | and sprouted) |
| Mung dal | Easy to digest lentil |
| Sunflower Seeds | Vitamin E (preferred organic and sprouted) |
| Rolled Oats | Calcium balance |
| Green Barley | Chlorophyll and anti-oxidants and SOD (super oxide Dismutase) |
| Fenugreek | Contains iron, Vitamin A, flavonoids, steroidal saponins |
| Spelt | High in B1 vitamin (preferred organic and sprouted) |
| Buck Wheat | High in rutin (preferred organic and sprouted) |
| Apples | High in pectin and quercitin. Typically, organic and essentially |
| | fresh. |
| Banana | High in potassium. Typically, fresh and organic. |
| Oranges | Vitamin C and bioflavonoids |
| Lemon | Citric acid and bioflavonoids |
| Ginger | Vaso dilator (typically liquid extract) |
| Pumpkin | High in amino acid arginine (preferred organic and sprouted) |
| Raw Honey | Provides quick energy and also assists in holding end product |
| | together |
| Blueberry Phenolic | Anti-Oxidant |
| Compound | |
| Papaya | Contains high amounts of papain |
| Unfiltered Honey | Palatability |
| Mango | Contains Tartaric acid, malic acid, citric acid and Vitamin C. |
| | Typically fresh and organic (without pit). |
| Cinnamon | Soothes digestive tract. Dry. |
| Celtic Sea Salt | Contains sodium and chloride. Dry. |
| Bromelain | Proteolytic enzyme. Dry. |
| Maca Root Powder | Peruvian ginseng. Dry. |
| Sensoril ® | Potent Ashwaganda. Dry. |
| Suntheanine ® | Extract from black tea |
| Boswellian Extract | Anti-inflammatory |
| Turmeric Extract | Anti-inflammatory (typically liquid form) |
| Curcumin | Active constituent of tumeric; antitumor, antioxidant, anti-amyloid |
| | and anti-inflammatory properties |
| White Willow Bark | Contains normally occurring salicin |
| Extract | |
| Dark Red Grapes | High in phenolic compounds. Typically organic and fresh. |
| Organic freeze dried | One of the richest sources of Glutamine, which helps to maintain |
| green cabbage | the gut mucosal integrity and function |
| Organic freeze dried | High in flavonoids and insoluble fiber |
| whole apple powder | |
| Active aloe | A micro-crystallized form of aloe juice with a pH of 8.6 (approx.) |
| | providing an alkaline buffer capacity. Can also help as an anti- |
| | inflammatory agent on gastric ulcers |
| Mycofusion ™ | Traditional Chinese medicine relies on mushrooms to support a |
| Mushrooms | variety of body-system imbalances. Cordyceps is a particular |
| (Cordyceps) | fungi used by eastern practitioners for proper liver functioning and |
| | health. Studies in China have demonstrated Cordyceps |
| | supportive action on the liver. Dry. |
| Mycofusion ™ | Among the most clinically studied whole foods in eastern medicine |
| Mushrooms (Reishi, | for immune healing properties. These medicinal fungi possess |
| Maitake, Shiitake) | beta 1,3-D glucan, a type of polysaccharide that stimulates |
| | immune cells. Shiitake has been licensed as an anticancer drug |
| | by the Japanese FDA. Maitake extract is currently undergoing its |
| | third clinical trial for breast cancer at New York's Sloan Kettering |
| | Hospital and Reishi has been found to inhibit growth and |
| | proliferation of prostate cancer cells. Research suggests that |
| | these mushrooms stimulate the body's production of the potent |
| | free radical scavenger, SOD. Dry. |
| Goji Berries | This traditional fruit of Tibet and China is high in carotenoids and |
| | vitamin C, known in western medicine for their immune system |
| | support. Recent research from University College Cork in Ireland |
| | showed that bioaccessibility of carotenoids is greater from this fruit |
| | than dark green vegetables. (preferred substantially fresh) |
| Ghee | A foundation food of Ayurvedic healing. It is essentially butter oil |
| | without the lactose and other milk solids. It is considered a |
| | balancing food for the entire body-system, and is recommended |
| | specifically for digestion, to aid in balancing excess stomach acid |
| | and maintain/repair the mucus lining of the stomach. Typically |
| | liquid form. |

One or more of the ingredients of Table 1, along with other possible ingredients discussed below, may be combined to form various embodiments of whole food equine products, with different embodiments for differing needs for equine applications. The various products comprise three formulation categories: Daily Formulas, Target Response and Treats.

Daily Formulas may be used as part of a regular ongoing daily diet for horses. Daily Formulas include several different formulations, generally known as:

Spark Plug Eq.: this formulation activates feed digestion and absorption and improves the effectiveness of supplements and medications overall.

Pre Performance Eq.: this formulation targets improved stamina in horses.

Post Performance Eq.: this formulation provides super green food and electrolytes for general health of horses. This formulation is rich in chlorophyll, vitamins, minerals and amino acids, fiber, enzymes, phytonutrients and alkaline minerals.

Whole Horse Spring/Summer Eq.: this formulation provides whole food anti-oxidants, purple kculi corn (typically Peruvian) as an anti-oxidant (preferred sprouted and organic) and Quercitin for allergies including inhibiting histamine release.

Whole Horse Fall/Winter Eq.: this formulation provides body-warming nutrients.

Target Response: this category of formulations target particular desired responses from horses. Target Response formulas include seven different formulations, generally known as:

Furnace Eq.: this formulation increases circulation and provides the substrate for nitric oxide production.

Comfort Zone A Eq.: this formulation supports reduction in pain and inflammation for acute cases of injury and stress, featuring tumeric.

Equilibrium Eq.: this formulation helps reduce physical and mental stress, featuring the patented nutraceutical Sensoril®, derived from asgawanda.

Liqatend Eq.: this formulation supports connective tissue healing and repair.

Energee Eq.: increases overall energy of horses and promotes increased athletic ability.

True Balance Eq: helps reduce the effect of stress on three major body systems: digestive/intestinal, liver and immune.

Tum-Ease Eq: helps reduce the effect of stomach acid production on intestinal mucosa.

MACA Impulsar Eq: a high potency formulae to meet high energy performance demands.

Treats category of formulations targeting particular ailments and may be used as required and include:

Whole Horse Wafers: formula is same as Whole Horse Spring/Summer Eq., but half the weight—in this form, it is a “treat.”

### FORMULATIONS

The ingredients for the various formulations (or matrices) are provided with preferred effective amounts of the selected ingredients from Table 1 to produce desired results and/or effects. Although the amounts of each ingredient used in the following formulations are described as the approximate preferred amounts, the amounts by weight (or specification as described) of each ingredient shown in each formulation (or matrix) may vary independently with ranges for any specific ingredient in ranges including about +/−5%, about +/−10%, and a non-preferred range of about +/−50% percent, from the amounts described below. The total amount or weight of a batch of formulation may also vary within the ranges of +/−5%, +/−10% or /−20%. The exemplary amounts of each ingredient shown below provide a representative formation (or alternate formulation) and the actual amounts of the formulation ingredients may be scaled as necessary to produce the respective formulation in mass quantities, preferably retaining the described ratio of ingredients, as a skilled artisan would recognize. A first formulation below is given in cups. A second alternate formulation below may be conveyed in ounces. The formulations below may also be expressed as a percentage (or parts) of the total weight and/or volume for any given formulation.

Spark Plug Eq. Formulation (yields about 91, 1½″1½″ bars):

| | | Approx. % |
|---|---|---|
| Ingredient | Amount | Total Weight |
| 2 cups sprouted Quinoa | 16 oz. | 17.78 |
| 2 cups organic oat bran | 10 oz. | 11.11 |
| 1 cup sprouted mung beans | 8 oz. | 8.89 |
| ½ large papaya | 2 lbs 12 oz. | 48.89 |
| ⅛ cup unfiltered honey | 4 oz. | 4.44 |
| Approx. Total Weight: | 90 oz. | |

Furnace Eq. Formulation (yields about 240, 1½″ 1½″ bars):

| | | | Approx. % |
|---|---|---|---|
| Ingredient | Amount | | Total Weight |
| ¼ cup dried ginger powder | 11 | oz. | 6.85 |
| 1 cup sprouted Quinoa | 8 | oz. | 4.98 |
| 1 cup sprouted Kamut berries | 8 | oz. | 4.98 |
| 1 and ½ cup oat bran | 15 | oz. | 9.35 |
| 1 and ½ cup wheat germ | 9 | oz. | 5.61 |
| 2 cups sprouted pumpkin seed | 16 | oz. | 9.97 |
| 2 cups sprouted sesame seed | 10 | oz. | 6.22 |
| 3 bananas | 42 | oz. | 26.17 |
| 2 whole oranges (including rind) | 21.5 | oz. | 13.40 |
| 1 whole lemon (including rind) | 11.5 | oz. | 7.16 |
| 1 Tablespoon raw honey | 2.5 | oz. | 1.56 |
| ¼ cup Ginger extract | 2 | oz. | 1.25 |
| ½ cup yeast | 4 | oz. | 2.49 |
| Approx. Total Weight: | 160.50 | oz. | |

Energee Eq. (yields about 70, 1½″×1½″ bars):

| | | | Approx. % |
|---|---|---|---|
| Ingredient | Amount | | Total Weight |
| 1 cup sprouted Amaranth | 8 | oz. | 9.11 |
| 1 cup sprouted mung beans | 8 | oz. | 9.11 |
| 1 cup oat bran | 10 | oz. | 11.39 |
| 1 cup wheat germ | 6 | oz. | 6.83 |
| 1 banana | 16 | oz. | 18.22 |
| 1 whole orange (including skin) | 10.3 | oz. | 11.73 |
| 1 whole lemon (including skin) | 11.5 | oz. | 13.10 |
| 1 cup sesame seed | 5 | oz. | 5.69 |
| ⅓ cup wheat grass | 8 | oz. | 9.11 |
| 2 tablespoons raw honey | 5 | oz. | 5.69 |
| Approx. Total Weight: | 87.8 | oz. | |

Whole Horse Wafers (yields about 308, 1½″×1½″ bars—typically thinner):

| | | | Approx. % |
|---|---|---|---|
| Ingredient | Amount | | Total Weight |
| 1 cup sprouted millet | 4.25 | oz. | 6.62 |
| 1 cup oat bran | 2.5 | oz. | 3.89 |
| 1 cup wheat bran | 2 | oz. | 3.12 |
| 1 cup sprouted purple Kculi Corn | 4 | oz. | 6.23 |
| 2 whole lemons | 11.5 | oz. | 17.91 |
| 2 whole oranges | 10.75 | oz. | 16.74 |
| 3 whole apples | 21 | oz. | 32.71 |
| 1 cup sprouted flax | 1.7 | oz. | 2.65 |
| ½ cup Blueberry Phenolic compound | 4 | oz. | 6.23 |
| 2 tablespoons raw honey | 2.5 | oz. | 3.89 |
| Approx. Total Weight: | 64.2 | oz. | |

Pre Performance Eq. (yields about 150, 1½″×1½″ bars):

| | | | Approx. % |
|---|---|---|---|
| Ingredient | Amount | | Total Weight |
| 1 cup sprouted Amaranth | 8 | oz. | 7.96 |
| ⅓ green barley | 2.25 | oz. | 2.24 |
| 1 cup sprouted Kamut wheat | 8 | oz. | 7.96 |
| berries | | | |
| ½ cup parsley | 1.25 | oz. | 1.24 |
| ½ cup oat bran | 5 | oz. | 4.98 |
| ½ cup sprouted mung beans | 4 | oz. | 3.98 |
| 1 cup sprouted sunflower seeds | 16 | oz. | 15.92 |
| 2 bananas | 28 | oz. | 27.86 |
| 2 whole lemons | 23 | oz. | 22.89 |
| 2 tablespoons raw honey | 5 | oz. | 4.98 |
| Approx. Total Weight: | 100.5 | oz. | |

Post Performance Eq. (yields about 114, 1½″ 1½″ bars):

| | | | Approx. % |
|---|---|---|---|
| Ingredient | Amount | | Total Weight |
| 1 cup oat bran | 10 | oz. | 12.44 |
| 1 cup soaked Fenugreek | 4 | oz. | 4.98 |
| 10 teaspoons wheat grass | 2.64 | oz. | 3.28 |
| 1 cup sprouted Spelt | 1.3 | oz. | 1.62 |
| 1 cup wheat germ | 3 | oz. | 3.73 |
| 2 whole lemons | 23 | oz. | 28.61 |
| 1 whole mango | 26 | oz. | 32.34 |
| 1 cup sprouted flax seeds | 3.4 | oz. | 4.23 |
| 1 cup sprouted buckwheat | 1.3 | oz. | 1.62 |
| 2 teaspoons celtic sea salt | 1 | oz. | 1.24 |
| ¼ cup dry ginger | 2.25 | oz. | 2.80 |
| 1 tablespoon raw honey | 2.5 | oz. | 3.11 |
| Approx. Total Weight: | 80.39 | oz. | |

Ligatend Eq. (Yields about 250, 1½″×1½″ bars):

| | | | Approx. % |
|---|---|---|---|
| Ingredient | Amount | | Total Weight |
| 2 cups oat bran | 10 | oz. | 7.32 |
| 2 cups wheat germ | 6 | oz. | 4.39 |
| 1 cup sprouted millet | 8.5 | oz. | 6.23 |
| ½ cup parsley | 3 | oz. | 2.20 |
| ½ cup garlic powder | 3.5 | oz. | 2.56 |
| ⅓ cup wheatgrass | 2.64 | oz. | 1.93 |
| 3 whole apples | 42 | oz. | 30.76 |
| 1 banana | 14 | oz. | 10.25 |
| 1 lb red or Concord grapes | 22 | oz. | 16.11 |
| 2 whole oranges | 21.5 | oz. | 15.75 |
| 1 cup sprouted flax | 3.4 | oz. | 2.49 |
| Approx. Total Weight: | 136.54 | oz. | |

Whole Horse Summer Eq. (yields about 202, 1½″×1½″ bars):

| | | | Approx. % |
|---|---|---|---|
| Ingredient | Amount | | Total Weight |
| 1 cup sprouted millet | 8.5 | oz. | 6.62 |
| 1 cup oat bran | 5 | oz. | 3.89 |
| 1 cup wheat bran | 4 | oz. | 3.12 |
| 1 cup sprouted purple Kculi Corn | 8 | oz. | 6.23 |
| 2 whole lemons | 23 | oz. | 17.91 |
| 2 whole oranges | 21.5 | oz. | 16.74 |
| 3 whole apples | 42 | oz. | 32.71 |
| 1 cup sprouted flax | 3.4 | oz. | 2.65 |
| ½ cup Blueberry Phenolic compound | 8 | oz. | 6.23 |
| 2 tablespoons raw honey | 5 | oz. | 3.89 |
| Approx. Total Weight: | 128.40 | oz. | |

TRUE BALANCE EQ (yields about 225, 1½″×1½″ bars):

| | | | Approx. % |
|---|---|---|---|
| Ingredient | Amount | | Total Weight |
| 3 Cups sprouted sunflower seeds | 24 | oz. | 9.05 |
| 3 cups sprouted pumpkin seeds | 24 | oz. | 9.05 |
| 3 cups sprouted flax seeds | 9.12 | oz. | 3.44 |
| 3 cups sprouted mung dal | 24 | oz. | 9.05 |
| 2 cups sprouted amaranth | 16 | oz. | 6.04 |
| 2 cups sprouted fenugreek | 8 | oz. | 3.02 |
| 4 cups diced cabbage | 32 | oz. | 12.07 |
| 2 cups apple powder | 16 | oz. | 6.04 |
| 2 cups goji berries | 18 | oz. | 6.79 |
| 2 cups brewers yeast | 16 | oz. | 6.04 |
| 2¼ cups bio-calcium | 18 | oz. | 6.79 |
| 1½ cups oat bran | 8 | oz. | 3.02 |
| 1 cup parsley | 2 | oz. | .75 |
| 1½ cups wheat germ | 9 | oz. | 3.39 |
| ½ cup ghee | 4 | oz. | 1.51 |
| ⅓ cup plus 8 tspns. mushrooms | 3 | oz. | 1.13 |
| 8½ tspns. aloe | 2 | oz. | .75 |
| 3 apples | 28 | oz. | 10.56 |
| ½ cup organic apple juice | 4 | oz. | 1.51 |
| Approx. Total Weight: | 265.12 | oz. | |

TUM-EASE EQ (Yields about 215, 1½″×1½″ bars):

| | | | Approx. % |
|---|---|---|---|
| Ingredient | Amount | | Total Weight |
| 4 cups sprouted amaranth | 32 | oz. | 17.77 |
| 2 cups sprouted mung dal | 8 | oz. | 4.44 |
| 3 cups sprouted sunflower seeds | 24 | oz. | 13.33 |
| 2 cups sprouted flax seed | 6.10 | oz. | 3.39 |
| 2 cups diced green cabbage | 8 | oz. | 4.44 |
| 6 cups oat bran | 30 | oz. | 16.66 |
| 1 cup apple powder | 8 | oz. | 4.44 |
| 7½ tspns. aloe | 4 | oz. | 2.22 |
| 2 apples | 28 | oz. | 15.55 |
| ½ papaya | 2 | lbs. | 17.77 |
| Approx. Total Weight: | 180.1 | oz. | |

COMFORT ZONE A EQ (Yields about 300, 1½″×1½″ bars):

| | | | Approx. % |
|---|---|---|---|
| Ingredient | Amount | | Total Weight |
| 3 cups sprouted buckwheat | 30 | oz. | 9.62 |
| 3 cups sprouted pumpkin | 24 | oz. | 7.70 |
| 2 cups sprouted flax | 6.8 | oz. | 2.18 |
| ½ cup tumeric extract | 4 | oz. | 1.28 |
| ½ cup white willow extract | 4 | oz. | 1.28 |
| 10 cups oat bran | 50 | oz. | 16.04 |
| 5 cups wheat germ | 15 | oz. | 4.82 |
| 2 cups bromlain | 13 | oz. | 4.17 |
| ½ cup dry ginger | 3 | oz. | .96 |
| 3 apples | 42 | oz. | 13.47 |
| 3 bananas | 70 | oz. | 22.45 |
| ½ lb dark grapes | 8 | oz. | 2.56 |
| ½ papaya | 2 | lbs. | 10.26 |
| 4 tblspns raw honey | 10 | oz. | 3.21 |
| Approx. Total Weight: | 311.80 | oz. | |

MACA IMPULSAR EQ (Yields about 213, 1½″1½″ bars):

| | | | Approx. % |
|---|---|---|---|
| Ingredient | Amount | | Total Weight |
| 4 cups sprouted amaranth | 32 | oz. | 11.00 |
| 4 cups oat bran | 20 | oz. | 6.87 |
| 4 cups maca | 32 | oz. | 11.00 |
| 2 cups spirilina | 16 | oz. | 5.50 |
| 2 cups brewers yeast | 16 | oz. | 5.50 |
| 4 lemons | 44 | oz. | 15.12 |
| 4 oranges | 84 | oz. | 28.87 |
| ½ papaya | 2 | lbs. | 11.00 |
| 6 tblspns raw honey | 15 | oz. | 5.15 |
| Approx. Total Weight: | 291 | oz. | |

EQUILIBRIUM EQ (Yields about 125 1½×1½″ bars):

| | | | Approx. % |
|---|---|---|---|
| Ingredient | Amount | | Total Weight |
| 2 cups sprouted buckwheat | 20 | oz. | 13.47 |
| 1 cup sprouted mung dal | 8 | oz. | 5.39 |
| 1 cup sprouted pumpkin | 8 | oz. | 5.39 |
| 6 cups oat bran | 30 | oz. | 20.20 |
| ½ cup plus 2½ tspns. | 3.5 | oz. | 2.36 |
| Sensoril ® | | | |
| 2 apples | 28 | oz. | 18.86 |
| 1 orange | 10 | oz. | 6.73 |
| 2 bananas | 26 | oz. | 17.51 |
| 6 tblspns raw honey | 15 | oz. | 10.10 |
| Approx. Total Weight: | 148.50 | oz. | |

WHOLE HORSE WINTER (Yields about 150 1½″×1½″ bars):

| | | | Approx. % |
|---|---|---|---|
| Ingredient | Amount | | Total Weight |
| 3 cups sunflower | 30 | oz. | 14.02 |
| 2 cups buckwheat | 20 | oz. | 9.35 |
| 1 cup mung dal | 8 | oz. | 3.74 |
| 4 cups oat bran | 25 | oz. | 11.68 |
| 1 cup hemp protein | 14 | oz. | 6.54 |
| 1 cup parsley | 2 | oz. | .93 |
| ½ cup ginger | 3 | oz. | 1.40 |
| 3 tblspns cinnamon | 1½ | oz. | .70 |
| 4 bananas | 56 | oz. | 26.17 |
| ½ papaya | 2 | lbs. | 14.95 |
| 1 orange | 10 | oz. | 4.67 |
| 5 tblspns raw honey | 12½ | oz. | 5.84 |
| Approx. Total Weight: | 214 | oz. | |

### Exemplary Process for Making Formulations

In order to make the various formulations as described previously, begin with the sprouted raw materials (e.g., frozen or fresh grains, seeds, grasses or vegetables). In varying embodiments, the sprouted material includes grains, seeds, grasses or vegetables that can be fed to livestock or equine animals. They include, but are not limited to, organic, certified organic and non GMO/GE of the following: organic amaranth, organic quinoa, organic kamut, organic oats, organic hull-less oats, organic spelt, organic sesame seeds, organic pumpkin seeds, organic sunflower seeds, organic mung dal, organic mung beans, organic green lentils, organic fenugreek, organic broccoli, organic alfalfa, organic buckwheat, organic hard winter wheat, organic wheat berries, organic flax seeds, organic wheat grass, organic barley, organic millet, organic purple corn, and blueberry phenolic powder.

Combine sprouted material from the oat family (or other basic grains such as wheat or barley). Add organic raw wheat germ and/or organic wheat bran and/or organic oat germ.

As required per individual formula: add other dry materials such as seeds, grasses, dried herbs and/or dried herbal extracts, yeast, roots, proteins which might include but are not limited to cayenne, celtic sea salt, dried organic ginger, organic dried flaxseed, organic hemp protein, spouted and organic quiona, organic rolled oats, organic powdered barley grass, organic powdered wheat grass, organic green kamut, bromelein, other dried herbs (such as milk thistle, tumeric, organic cinnamon from Sri Lanka or Vietnam, garlic, boswellia), Brewer's yeast, maca root. All dry materials can be organic, certified organic, non GMO/GE treated and can be imported or domestic.

In food processor (or other suitable blender, mixer, grinder) combine: bananas, and/or papaya, and/or fresh or frozen ground orange peels, and/or fresh or frozen orange pulp, and/or orange juice from concentrate or fresh squeezed, or fresh squeezed/frozen orange juice, and/or mango and/or apple, and/or fresh squeezed lemon, and/or fresh or frozen ground lemon peels, and/or lemon juice concentrate. All fruits and vegetables are organic or certified organic, typically non GMO or GE. In some embodiments, other fruit and vegetables can be substituted for the above mentioned fruit.

Add liquid herbal extracts per specific formula. The herbal extracts may include, and not limited to: ginger extract, willow bark extract, boswellia extract, bromelein extract, tumeric extract, milk thistle extract. All extracts are typically organic or certified organic, and typically non GMO or GE.

As needed per formula, add as appropriate: raw honey and/or unfiltered honey (all varieties of domestic or imported honey or processed honey can be substituted), agave nectar (either organic or non organic), and/or brown rice syrup. Any other suitable organic, certified organic or processed sweetener may be equivalently substituted for the above sweeteners, if necessary.

Add as needed per formula: (other organic, certified organic and non GMO or GE grains, seeds, beans, vegetables or grasses can be substituted for the following): organic sprouted sunflower seeds, and/or organic sprouted pumpkin seeds or soaked pumpkin seeds, and/or organic sprouted fenugreek seeds, and/or organic sprouted sesame seeds, and/or organic sprouted mung beans, and/or organic sprouted mung dal or soaked mung dal, and/or organic sprouted green lentils, and/or organic sprouted flax seeds, or soaked flax seeds, and/or organic sprouted broccoli seeds, and/or organic sprouted alfalfa, and/or organic sprouted wheat grass, and/or organic sprouted green barley.

Add as needed per specific formula: extra virgin olive oil (or domestic olive oil or any other organic, certified organic, non GMO or GE vegetable oil), organic, certified organic apple cider vinegar (or any other organic, certified organic and non GMO or GE fruit vinegar).

Blend batter until thoroughly mixed. For example, set a food processor to “1” for 2 minutes, then go to “pulse” until batter is thoroughly mixed. If a blender, mixer or other mechanical mixing device is used, these times are changed to achieve the same equivalent results.

Add batter to dry material. Stir, either by hand or mechanical means, to make sure batter and dry material blend well. Adjust beater on mixer and mix for two to five minutes. If using mechanical means to combine wet and dry materials, the timing of the mixing process will change, as appropriate, to achieve generally uniform mixture.

Remove from mixer and pour into Rotary Molder Drum System (or any other machine that can produce cookies and/or bars). Automatic cutting or forming mechanisms is preferred for forming the cookies or bars. But, the dough may be also be cut by hand using cookie/biscuit form, or other suitable instrument.

Dehydrate the cookies or bars (i.e., matrix) at 145 degrees (F) for approximately three hours; then 108 degrees (F) for approximately 10 hours (+/−20%). For some formulations, the temperature range may vary by as much as 10% (i.e., about 1%-10%), preferably no more than 5%. This relatively low temperature prevents damage to the effectiveness of the various ingredients. Depending on moisture content, it may be necessary to turn the dehydration trays (or the matrix) 180 degrees, and dehydrate for one to three more hours at 108 degrees (F). Dehydration should be monitored to achieve suitable dehydration depending on total initial moisture content, which can vary depending on the nature of the ingredients being used and/or formulation. The degradation process is meant to remove most of the water content, which typically results in a product having water content less than 3% of original water content. Preferably, the result is water content is less than 1% of original water content. However, a final water content of less than 4% of original water content would be acceptable.

Generally, dehydration should occur at a temperature to achieve the target water content (typically less than 1%, but may vary as described above) without destroying the biological effectiveness of any nutrient or ingredient.

A total weight reduction of each formulation due to the dehydration step varies from about 21% to about 42% total weight reduction, depending on the particular product and water content contributions of each ingredient. The variation in total weigh reduction for any given formulation due to dehydration may vary by as much as 10%, but preferably less than 5%, with a more preferred goal of less than 3% variation. Typical dehydration reduction percentage by weight (approximately) for each formulation is shown below in Table titled “Dehydration Reduction.”

TABLE
DEHYDRATION REDUCTION

| | Approx. % Total Weight Reduction |
|---|---|
| FORMULATION | Due to Dehydration Process |
| Spark Plug | 37.7 |
| Equilibrium | 31.1 |
| Furnace | 41.5 |
| Whole Horse Winter | 31.7 |
| Whole Horse Summer | 23.7 |
| Whole Horse Wafers | 23.7 |
| Comfort Zone | 27.3 |
| Ligatend | 21.2 |
| Pre Performance | 33.00 |
| Post Performance | 28.06 |
| Energee | 31.00 |
| Maca | 29.5 |
| True Balance | 31.8 |
| Tum Ease | 30.00 |

Dehydrators can be set at varying temperatures; therefore dehydrating time for each recipe can be altered/adjusted as to the needs of the material being dried.

Remove and package in plastic ziplock bags with moisture reduction packets, or other suitable storage packaging, then place in moisture proof plastic containers. Alternative methods of shipping the product might include but is not limited to: frozen, flash-frozen, vacuum packed, with or without moisture reducing packets or non-moisture proof plastic container, for shipping a food product for long or short distances.

### Test Results

Test results of each of the formulations herein have shown to be effective by actual application over time in test equine subjects. Many of these formulations herein are now being used by internationally recognized horse trainers and competitive teams, with favorable results for intended purpose of each formulation when administered over time. Exemplary results of representative formulations are described below, producing the effects as described. Some of these tests are described below.

Palatability tests were performed on nineteen test horses. All ingredients were tested in test horses with acceptable results, with no negative side-effects. Table INGREDIENTS TEST, shows results on acceptance of selected ingredients.

TABLE
INGREDIENTS TEST

| TEST | RESULTS |
|---|---|
| 1. Palatability of dehydrated sprouts | 100% acceptability from all 19 |
| | horses. |
| 2. Palatability of fresh sprouts | 100% acceptability from all 19 |
| | horses. |
| 3. Palatability of hydrated bars. | 95% acceptability from all 19 |
| | horses. |
| 4. Palatability of bananas | 100% acceptability from all 19 |
| | horses |
| 5. Palatability of papayas | 100% acceptability from all 19 |
| | horses. |

### Tests on Spark Plug Formulation

Spark Plug was tested over a six month period on 6 horses, each having two to four bars of Spark Plug per day. The only dietary change for the six months was the addition of Spark Plug to the diet. These horses were all high performance dressage horses. The horses names were: Peppie, Imager, Maestro, Solon, Lionheart, and Peter.

The horse Lionheart had a long record of recurring lameness's that required joint injections and systemic injections. Lionheart showed an immediate improvement after thirty days on Spark Plug.

The amount of commercial feed given per day to Maestro was reduced after one month on Spark Plug, leading to the probable conclusion that Spark Plug helped this horse utilize his feed better, and so the owner could reduce the amount of commercial feed given.

The horse Peppie showed a noticeable change in behavior after two months on Spark Plug. His groom during the test period noted that he was less cranky and irritable.

The horse Imager, who had a history of being picky and choosey about his feed, was noticeably less finicky after two months on Spark Plug. This stallion had a propensity to stop eating his commercial feed after several months, and the owner would have to switch to a different brand or type of feed. This pattern had been consistent with this horse for two years. Since Imager has been on Spark Plug, beyond the two months, the owner has not had to switch brands of feed.

The horse Peter showed no obvious changes on Spark Plug during the test period, but the owner did observe that after several months during the test, she thought the horse looked better than he ever had. However, how much this related to Spark Plug and how much to the horse's training is not clear.

The horse Solon, the youngest of the test horses (age 5), showed an improved attitude in his daily training sessions after three months testing on Spark Plug.

After six months total test period, five of the horses (Peppie, Maestro, Solon, Imager, Lionheart) showed a “glow” in their appearance as compared to the other fourteen horses not included in the Spark Plug test, at this particular barn. The horse Peter is not stabled at the same facility, and so was not compared. At the request of the test horses' owners, following the conclusion of the Spark Plug test/study, the test horses were continued on Spark Plug.

### Tests on Furnace Formulation:

Furnace was tested over an eight month period with three horses. The horses were Peppie, Chamberlain and Lionheart. All three horses had long histories of problems relating to the hoof. All three horses had periodic injections of the coffin joint and clinical veterinarian diagnosis of “inflammation of the bursa.” It had been recommended by the United States Equestrian Team (USET) veterinarian, that the horse Lionheart be “nerved” (i.e., nerves in the foot cut surgically to reduce his pain). The horse Peppie dropped out of this study after two weeks, because he refused to eat it, in general. The two remaining horses were on four bars of Furnace per day. Improvement in Lionheart was noted in two weeks. The same USET veterinarian, after seeing the horse 10 weeks after being on Furnace pronounced, “this product has done what none of my veterinary drugs could do.” The USET veterinarian concluded that the horse did not have to be surgically nerved.

Foot improvement in the horse Chamberlain (also under the USET veterinarian's care) was noticed after ten days of being on the product. After the eight months, Lionheart had maintained his soundness without further injections of the coffin joint. After eight months, Chamberlain had maintained his foot soundness without further injections of the coffin joint. Both horses continued with testing on the product, at the request of the owners. Chamberlain has increased his daily intake to 8 bars per day due to a heavy showing season and maintains his soundness.

### Tests on Furnace with Spark Plug for USET Veterinarian

The USET veterinarian requested one month's supply of Spark Plug and Furnace for a Grand Prix jumper in Florida competing on the big winter Florida circuit. The Grand Prix jumper had shut down in competition, for which there was no apparent reason. This horse showed moderate discomfort in the feet but did not respond to coffin joint injection.

After the one month test on Spark Plug and Furnace, the USET veterinarian observed that horse “was happy with himself.” The Grand Prix jumper went back to competition and stayed sound, without additional Spark Plug or Furnace.

### Tests of Furnace for Severe Navicular Horse

Furnace was tested for two weeks on a Quarter horse with severe Navicular Syndrome. This horse, of Richmond, Va., did not respond at all to four bars of Furnace per day. It was concluded that four bars of Furnace was not enough to produce efficacy in this severe foot problem.

### Tests on Energee

The first test on Energee was conducted at the Regional Dressage Championships in Lexington, Va. Two horses were initially tested: the stallion Neostan, and the mare Rowena. The stallion Neostan had a history of lethargy in competition.

Two bars of Energee were given to Neostan two hours before his Finals class, on a Friday. Another two bars were given thirty minutes before the class. The rider remarked that the horse had never felt so energetic. Neostan won the Finals class at the International level.

The second day Neostan was given four bars two hours before Finals class on Saturday night, and another two bars thirty minutes before the class. Rider remarked that Neostan had too much energy, and only finished in 6th place. Sunday, the third day, two bars were given two hours before the class and two bars thirty minutes before the class, and the rider remarked that the Neostan felt wonderful. Neostan won that Finals class.

The mare, Rowena, has a history of running out of steam at a show after the first class. Rowena was given two bars of Energee one hour before the second class, and owner remarked that the horse had plenty of power and energy to compete. This was repeated on the second day, and Rowena won both her classes.

Another this test was conducted for one month with the stallion Imager. Imager is a lazy stallion, and two bars of Energee was given to him one hour before his daily training session. Results were felt by owner immediately. Horse had more energy throughout daily training session.

Yet another test was conducted for one month on gelding Peter. Two bars one hour before work were given, and no noticeable results were found. Dosage was increased to four bars and only a slight improvement noted. This horse had previously been on small doses of anabolic steroids and had no noticeable results from that treatment either.

### Test Results on PrePerformance

Six horses were tested on Pre Performance for three months. Those horses were Lionheart, Rowena, Peppie, Maestro, Imager, and Kostolan. No other dietary changes were given to the horses. Horses were given two bars forty-five minutes to two hours before training. All six horses showed improvement in stamina as measured by their riders. All six horses developed increased muscle in their hindquarters. The systematic training of these horses combined with the formula can explain the increase in muscle mass.

### Test Results on Post Performance

Four horses were tested on Post Performance for two months. Those horses were Kostolan, Imager, Denny, Lionheart. Horses were given two bars immediately following workouts.

All four horses showed noticeable improvement in respiration and recovery after hard physical training. The horse Denny, with the least stamina and the greatest fatigue prior to the testing, showed improvement after three days (as measured by respiration and heartbeat). The stallion Imager, went from a recovery time of thirty minutes, to a recovery time of fifteen minutes after one month on the formula. Kostolan, decreased his recovery time from forty five minutes to thirty minutes after one month on the formula. The horse Lionheart decreased his recovery time from twenty five minutes to twenty minutes after one month. Lionheart is the oldest horse in the test group (age 17) and it is surmised that his age contributed to a slower percentage of improvement in minutes of recovery time. It should be noted that Lionheart was also the most fit horse in the study with the lowest recovery time to start with.

A second test was conducted at two dressage competitions. The Regional Dressage Championships in Lexington Va., and the Williamston Dressage Competition in Williamston, N.C. At the Regional Dressage Championships, two horses were tested over three days of competition. They were Gossamer and Rowena. Respiration and heart beat were not taken in this test, but instead relied on basic physical signs of recovery from stress (the horses' eyes, measurement of hydration by pinching the skin, inspection of the gums for signs of dehydration and stress). Water consumption and feces evaluation were conducted throughout the three days. Each horse was given two bars immediately following their competition class.

The horse Gossamer became noticeably more relaxed throughout the show. Whether this was due to the ingestion of the formula was not confirmed during this test. His water consumption increased (lack of sufficient hydration is a real danger to horses). No noticeable change in feces.

The horse Rowena demonstrated a remarkable response after the second day of ingesting the product. Water consumption increased, her feces showed increased systemic hydration, horse sweated evenly around neck, shoulders and under the saddle.

Test at the Williamsburg Dressage show with four horses: Rowena, Blu, Magic, and Kostolan. This test was a follow up to the Lexington dressage test, to see how Post Performance worked on an “as needed” basis. Only one of these horses (Rowena) had been on the product on a daily basis. Two bars were given immediately following competition class.

Rowena maintained her recovery time (thirty minutes) as she had been on the product for a month. The stress of the show did not affect her recovery time.

Blu responded after his first serving with an increase in water consumption throughout the day and night. Rider reported that by the second day the horse felt better than he normally did on the second day of a show. Magic responded by eating more hay than usual after his first serving. This horse has a tendency to back off his feed and hay when competing. Kostolan demonstrated far less fatigue, according to his rider, on the second day of the competition. No change in water consumption or in feces consistency was shown.

### Test Results on Whole Horse Winter

Test number one (Henderson, Kentucky): this test of the Whole Horse formulation palatability was conducted over two days in Henderson, Kentucky on a variety of ten horses of various breeds including: Arabian, Quarter horse, Warmblood, and Morgan. Two of the ten horses would not eat the bars. Of the remaining 8, one refused a second bar when offered. The remaining seven each consumed six bars over two days.

Test number two: this Whole Horse formulation test was conducted on five horses: Imager, Peppie, Solon, Maestro, and Lionheart. Although no obvious changes were noted in these horses, this formulation was not designed to produce obvious “therapeutic” affects, since this product is more general, similar to a multi vitamin. Four of the five horses were given two bars per day, and one horse was given four. The bars were given after the horses had cooled out after training and were ready to go back to their stalls. One horse, Peppie, loved the bars so much that if the groom did not give him the bars fast enough he would paw and stamp his feet until he got a bar in his mouth. Peppie has been a test horse for several formulas, but this is the first formula about which he became very demanding.

Test number three (San Antonio, Tex.): this was another Whole Horse formulation palatability test on 15 horses of various breeds including: Warmblood, Andulusian, Thoroughbred, Mustang, and Quarter horse. Only one horse refused to eat the pro-offered bars. The remaining horses found the bars to be very palatable. It should be noted that the owners/riders of all the test horses asked to keep their horses on the various formulas. One owner remarked quite seriously “I can't possibly train and compete without these formulas.”

### Test Results on Whole Horse Summer:

Test number one: two Warmblood geldings (Denny, Ramses) and one Thoroughbred gelding (Nitro). All three horses suffer from skin allergies in the summer (itchy skin, allergic reaction to insect bites).

All three horses were given two bars twice a day. At the end of one month, Denny and Nitro were not itching and scratching. Ramses still had some skin allergy hypersensitivity, but improvement overall. Ramses also suffers from Cushings Disease, so the adrenal stress may be too much for Whole Horse Summer to help balance.

Test number two: one Warmblood gelding (Pi) with an acute case of hives all over his body. Horse was given a shot of Dexamethasone (steroid) to combat the hives, then shipped ten hours in a trailer.

Hives were still active after three days. USET vet examined Pi and recommended vinegar rinses. The vet conveyed that it would take two weeks for the bumps to go away, since the horse did not respond to Dexamethasone. Thereafter, Pi was given four bars of Whole Horse Summer twice a day. By day four, most of the hives had disappeared, with the exception of bumps still on his hindquarters. After three more days all the hives had gone. Pi was not rinsed daily with vinegar during the efficacy test of Whole Horse Summer.

Test number three: one Warmblood gelding (Eastie) and one Quarterhorse (TK). Both aged horses, Eastie being 23 and TK age 26. Both horses were sensitive to pollens, which result in nasal discharge and runny eyes. Eastie was given four bars twice a day for two weeks, then two bars twice a day for another two weeks. After the initial two week period, the nasal discharge was barely detectable, although there was still some clear fluid running from both eyes. At the end of thirty days, the eyes were still running, but there was no nasal discharge at all. Eastie continued for another thirty days on Whole Horse Summer (two bars twice a day) and by the middle of the second month, there was no discharge from his eyes.

TK started on four bars twice a day for two weeks, and showed minimum reduction in nasal discharge. He continued for another two weeks on four bars twice a day with no improvement. We concluded that this lack of response was due to the fact that TK, like Ramses, suffers from Equine Cushings Disease.

### Test Results for Tum Ease:

Test number one: one Warmblood gelding (Lionhart) who has a long history of being treated for intestinal ulcers. This horse demonstrates classic ulcer symptoms when he is stressed: he goes off his feed, is cranky and irritable, and refuses treats. Normal treatment of his ulcers has been with the drug Omeprazole (Gastro Guard). Lionheart was given two bars of Tum Ease before each grain meal, and in twelve days the horse returned to eating with gusto, and eating his treats. The owner reported that he had a “happy” look in his eyes again.

Test number two: two Warmblood geldings (Pi and Corinthian). Both exhibited ulcer symptoms while under saddle (teeth grinding, girthiness while being saddled, reluctance to work). Both horses were given two Turn Ease before tacking up, and two Turn Ease after being ridden. The rider of Pi reported that in five days the horse was much more settled and did not grind his teeth at all. Rider of Corinthian reported that after seven days Corinthian was no longer girthy and sensitive. Both horses continue to train on Tum Ease and neither horse has had to be put on the expensive ulcer drug Omeprazole.

Test number three: three Warmblood geldings (Chamberlain, Peppie and Mo), and one Warmblood stallion (Imager). Test to see effectiveness of Turn Ease in a competition environment, which produces increased stomach acid in horses, due to stress. All four horses normally are given Omeprazole every day during competition. At the end of the three days of competition, Chamberlain and Peppie exhibited ulcer symptoms, while Mo and Imager did not.

Test number four: two Warmblood horses (Chamberlain and Peppie) were given two bars of Turn Ease before getting on the trailer to go to a competition, and two bars when they arrived. This was repeated when the horses were shipped home. Owners reported both horses shipped better than before, and, in fact, Peppie, who is a chronic trailer kicker, hardly kicked the entire trip.

Test number five: two Warmblood horses (Chamberlain and Peppie) were given half a tube of Omeprazole before going to a competition. During the competition they were give two bars of Tum Ease before being ridden and immediately after being ridden. The horses were then given Omeprazole when they returned from the two-day show. Both riders reported their horses exhibited no ulcer signs the entire weekend. Normally these two horses would be given Omeprazole every day of the competition (half a tube). The cost of Omeprazole ($39.00 per tube) makes this drug especially expensive for many horse owners. The riders were very pleased that they didn't have to use so much Omeprazole to help their horses through the stress of competition.

Test number six: six year old Arabian mare in San Diego, Calif. Exhibits classic ulcer symptoms when being ridden. Owner used two bars of Tum Ease before riding, and two bars of Tum Ease after riding. The owner reported significant improvement in mare's attitude under saddle within three days.

### Test Results for True Balance:

Test number one: three Warmblood geldings (Pi, Granusch and Denny). All horses had been treated for ulcers a year before with Omeprazole. Pi refused to eat the bars. Granusch and Denny, however, gobbled up the bars. Granusch and Denny were given two bars twice a day for thirty days. Groom reported that Denny and Granusch were not nearly as fussy with their hay and feed after the full month on True Balance. The owners of Denny and Granusch reported that their horses' coats had improved, and in Granusch's case, the quality of his hooves had improved. This was also confirmed by the farrier. The riders reported that both horses were less grumpy under saddle.

Test number two: two Andulusian horses in Arkansas (Baria and Cubby). Baria has a history of laminitic episodes, and gas colics. Cubby is quite aged (32) and suffers from years of NSAIDS (non-steroidal anti-inflammatory) therapy. Cubby also is an extremely fussy eater, and needs to gain weight. Both horses were put on two bars twice a day for thirty days. Baria showed no obvious signs of improvement until week three, when her owner reported that the horse was much more eager to eat her hay and grain and had no gas colic episodes. Cubby showed noticeable improvement by week two as evidenced by galloping around his pasture—something he hadn't done in a year. At the owner's request, both horses continued on True Balance for an additional thirty days, in which time Baria had no colic episodes and the owner was able to increase Cubby's hay and grain consumption.

Test number three: one Warmblood gelding (Eastie) and one thoroughbred gelding (Nitro). Eastie is a former ulcer horse, but has exhibited no symptoms in four years. However, he suffered a severe trauma to his left front flexor tendon so it was decided to test True Balance on him. Nitro is a chronic gastric ulcer horse, who has been on calcium carbonate for a year to buffer his stomach acid. Both horses were given two bars twice a day. Eastie's urine production increased within five days on True Balance. This is likely due to the liver support of the mushrooms in True Balance which help detoxify the liver. Since Eastie was on high antibiotic therapy for the infection in his tendon, it was concluded that True Balance assisted his body in processing and getting rid of toxins. Eastie also consumed more water during the thirty day test. Since Eastie has had impaction colic episodes, the veterinarian was pleased to see an increase in water consumption.

Nitro had been taken off calcium carbonate for thirty days prior to testing of True Balance. After thirty days, his owner reported that the horse's coat quality had improved, and that he was eating all his grain and hay (where previously he would leave some of his grain and a lot of his hay).

### Test Results on Ligatend:

Test number one: a Warmblood gelding (Solon) diagnosed through ultrasound with a strained right hind suspensory. Diagnosis: two months stall rest, then three months of walk and trot under saddle.

Started horse on two bars twice a day as soon as diagnoses was confirmed with ultrasound. The vet re-ultrasounded Solon after thirty days and pronounced the suspensory ligament healed. Horse could start back walking after only thirty days stall rest. Results confirmed by ultrasound. Solon continued on Ligatend for another thirty days.

Test number two: a Warmblood gelding (Mo) diagnosed with a weak right hind suspensory. Treatment was icing twice a day, light work, and minimum turn out. Mo was given Ligatend (two bars twice a day) and vet rechecked horse in thirty days. The vet told owner that the ligament never felt better. The vet recommended that the horse stay on Ligatend.

Test number three: Warmblood mare (Rowena) diagnosed with torn collateral ligament. The horse proceeded to get severe attack of laminitis, and was shipped to vet clinic in North Carolina. The equine podiatrist and the attending vet gave poor prognosis for the mare's recovery to full work. Rowena was given Ligatend (two bars twice a day) with two bars of Furnace twice a day for four months. After four months at the vet hospital, the horse was sound and able to start back in work. Podiatrist and vet never expected this horse to recover full soundness. Horse is scheduled to begin competing again.

### Test Results on Maca Impulsar:

Two tests were performed on Maca Impulsar. The first test was on two imported Dutch Friesan geldings (one age 11, the other age 12). Both horses suffer from heat and humidity in the summer, resulting in lack of the necessary energy to train and compete in Dressage.

The eleven year old was given two bars two hours before exercise. After four days, the rider reported the horse showed no change. Dosage was increased to four bars and after two days, the rider reported the horse had much improved energy levels.

The twelve year old Friesan horse was also given two bars two hours before exercise and the rider reported that by day four the horse was much more energetic.

Test number two was given to three German Warmblood stallions, all competing in Dressage. Two bars were given two hours before training. All three riders reported an improvement after day two.

A possible conclusion might be made that the Friesan horses with much colder blood lines (no Thoroughbred or Arabian bloodlines) took longer to gain the benefits of the Peruvian plant, Maca. The Warmblood stallions, all of whom have Thoroughbred or Anglo/Thoroughbred ancestry, were able to utilize Maca more efficiently in a shorter amount of time.

### Test Results on Equilibrium EQ:

Test number one: a total of 24 horses were tested over a period of four months on Equilibrium EQ. Twenty-three of the 24 horses were all European Warmblood horses. One horse was a thoroughbred.

Of the 24 total test horses, 20 were tested for a reduction of stress and anxiety in competition and training environments. Of the 20 horses tested, only two refused to eat Equilibrium EQ. The remaining 18 were given two bars before being ridden. Ten of these horses showed a noticeable improvement (as described by the riders) in being able to focus more on their work. The other eight horses were then given two bars twice a day. Seven of these horses demonstrated a reduction in nervousness and stress, and greater concentration and less spookiness than before. One horse did not respond to two bars twice a day and was subsequently given four bars twice a day which produced a noticeably calmer animal.

The remaining four horses were tested for Equilibrium's use in lay-up situations where horses are confined to their stalls for weeks or months at a time for medical reasons. Two of the four horses stayed calm and unstressed during their eight-week stall confinement on two bars twice a day. One horse had to be given three bars three times a day to stay calm and unstressed. The fourth horse ate the bars well for the first week, showed noticeable improvement in the reduction of stress, then refused to eat the bars anymore.

Test number two: six Warmblood geldings were tested for the reduction of stress in trailering and/or monthly farrier work. Three of these geldings were given two bars two hours before trailering. Two of these horses are chronic trailer kickers, whose stress levels include kicking repeatedly in the trailer. On Equilibrium, the kicking was reduced in one horse, and the other did not kick at all. The third gelding had to be given four bars prior to trailering for the stress levels to be reduced.

The two Warmblood geldings tested for farrier work have a long history of being unable to stand quietly while their feet are being trimmed. The owners often tranquilize these horses for the safety of the horse and the blacksmith.

One of the geldings needed four bars of Equilibrium (two bars an hour before the blacksmith arrived and an additional two bars halfway through the trimming).

The second gelding was given four bars before the blacksmith arrived and stood quietly the entire session. The blacksmith reported that neither gelding had ever been so good to shoe, except on a pharmaceutical tranquilizer.

Possible conclusion on Equilibrium EQ: Equilibrium clearly supports the reduction of stress for horses confined due to illness, and helps reduce anxiety in specific anxiety-producing situations.

While the invention has been described in terms of exemplary embodiments, those skilled in the art will recognize that the invention can be practiced with modifications in the spirit and scope of the appended claims. These examples given above are merely illustrative and are not meant to be an exhaustive list of all possible designs, embodiments, applications or modifications of the invention.

What is claimed:

1. A solid equine-edible matrix having a discrete shaped form for the delivery of nutrients wherein said solid equine-edible matrix provides nutrients suitable for an equine diet that comprises a compressed mixture of a grain product and nutrients, said matrix having a maximum moisture content of less than 5% by weight, said matrix having been produced by the process of mixing said grain products with said nutrients; and thereafter removing moisture to a level at or below said maximum moisture content by exposure of said shaped object to a prolonged exposure at a temperature not higher than about 145° F., whereby moisture is removed from said shaped object resulting in said solid matrix having a discrete shaped form, whereby said solid matrix having a discrete shaped form contains an enhanced level of nutrients.

2. The solid equine-edible matrix of claim 1, wherein the maximum moisture content is less than 1% by weight.

3. The solid equine-edible matrix of claim 1, wherein a weight of the removed moisture ranges from about 21% to about 42% of an initial weight of the matrix.

4. The solid equine-edible matrix of claim 1, wherein the temperature varies in the range of about 1% to about 10%.

5. The solid equine-edible matrix of claim 1, wherein the step for removing moisture includes dehydrating the matrix at about 145° F. for approximately three hours, then dehydrating the matrix at about 108° F. for approximately 10 hours.

6. The solid equine-edible matrix of claim 5, wherein the step for removing moisture further includes turning the matrix 180 degrees, and dehydrating for about one to three more hours at about 108° F.

7. The solid equine-edible matrix of claim 1, wherein said matrix comprises the following ingredients: sprouted Quinoa, organic oat bran, sprouted mung beans, sprouted sunflower, papaya and honey.

8. The solid equine-edible matrix of claim 1, wherein said matrix comprises the following ingredients: dried ginger powder, sprouted Quinoa, sprouted Kamut berries, oat bran, wheat germ, sprouted pumpkin seed, sprouted sesame seed, bananas, oranges, lemon, honey, Ginger extract and yeast.

9. The solid equine-edible matrix of claim 1, wherein said matrix comprises the following ingredients: sprouted Amaranth, sprouted mung beans, oat bran, wheat germ, banana, orange, lemon, sesame seed, wheat grass and honey.

10. The solid equine-edible matrix of claim 1, wherein said matrix comprises the following ingredients: oat bran, rolled oats, yeast, wheat germ, sprouted sunflower seeds, bananas and cinnamon.

11. The solid equine-edible matrix of claim 1, wherein said matrix comprises the following ingredients: sprouted millet, oat bran, wheat bran, sprouted purple Kculi corn, lemons, oranges, apples, sprouted flax, Blueberry Phenolic compound, honey.

12. The solid equine-edible matrix of claim 1, wherein said matrix comprises the following ingredients: sprouted Amaranth, green barley, sprouted Kamut berries, parsley, oat bran, sprouted mung beans, sprouted sunflower seeds, bananas, lemons and honey.

13. The solid equine-edible matrix of claim 1, wherein said matrix comprises the following ingredients: oat bran, soaked Fenugreek seeds, wheat grass, sprouted Spelt, wheat germ, lemons, mango, sprouted flax seeds, sprouted buckwheat, celtic sea salt, dry ginger and honey.

14. The solid equine-edible matrix of claim 1, wherein said matrix comprises the following ingredients: oat bran, wheat germ, sprouted millet, parsley, garlic powder, wheatgrass, apples, banana, red or Concord grapes, oranges and sprouted flax.

15. The solid equine-edible matrix of claim 1, wherein said matrix comprises the following ingredients: sprouted millet, oat bran, wheat bran, sprouted purple Kculi Corn, lemons, oranges, apples, sprouted flax, Blueberry Phenolic compound and honey.

16. The solid equine-edible matrix of claim 1, wherein said matrix comprises the following ingredients: sprouted sunflower seeds, sprouted pumpkin seeds, sprouted flax seeds, sprouted mung dal, sprouted amaranth, sprouted fenugreek, diced cabbage, apple powder, goji berries, brewers yeast, bio-calcium, oat bran, parsley, wheat germ, ghee, mushrooms, aloe, apples and apple juice.

17. The solid equine-edible matrix of claim 1, wherein said matrix comprises the following ingredients:

18. The solid equine-edible matrix of claim 1, wherein said matrix comprises the following ingredients: sprouted amaranth, sprouted mung dal, sprouted sunflower seeds, sprouted flax seed, diced green cabbage, oat bran, apple powder, aloe, apples and papaya.

19. The solid equine-edible matrix of claim 1, wherein said matrix comprises the following ingredients: sprouted buckwheat, sprouted pumpkin, sprouted flax, tumeric extract, while willow extract, oat bran, wheat germ, bromlain, dry ginger, apples, bananas, dark grapes, papaya and honey.

20. The solid equine-edible matrix of claim 1, wherein said matrix comprises the following ingredients: sprouted amaranth, oat bran, maca, spirilina, brewers yeast, lemons, oranges, papaya and honey.

21. The solid equine-edible matrix of claim 1, wherein said matrix comprises the following ingredients: sprouted buckwheat, sprouted mung dal, sprouted pumpkin, oat bran, Sensoril®, apples, orange, bananas and honey.

22. The solid equine-edible matrix of claim 1, wherein said matrix comprises the following ingredients: sunflower, buckwheat, mung dal, oat bran, hemp protein, parsley, ginger, cinnamon, bananas, papaya, orange and honey.

23. A method of equine treatment for treating digestion and absorption, comprising the step of administering an equine supplement over time, the supplement comprising the following ingredients: sprouted Quinoa, organic oat bran, sprouted mung beans, sprouted sunflower, papaya and honey.

24. The method of claim 23, wherein a weight of any of the ingredients varies as a percentage of the total weight of the supplement within the range of +/−5%.

25. The method of claim 23, wherein a weight of any of the ingredients varies as a percentage of the total weight of the supplement within the range of one of: +/−10% and +/−50%.

26. A method of equine treatment for increasing circulation and providing a substrate for nitric oxide production, comprising the step of administering an equine supplement over time, the supplement comprising the following ingredients: dried ginger powder, sprouted Quinoa, sprouted Kamut berries, oat bran, wheat germ, sprouted pumpkin seed, sprouted sesame seed, bananas, oranges, lemon, honey, Ginger extract and yeast.

27. The method of claim 26, wherein a weight of any of the ingredients varies as a percentage of the total weight of the supplement within the range of +/−5%.

28. The method of claim 26, wherein a weight of any of the ingredients varies as a percentage of the total weight of the supplement within the range of one of: +/−10% and +/−50%.

29. A method of equine treatment for supporting connective tissue healing and repair, comprising the step of administering an equine supplement over time, the supplement comprising the following ingredients: oat bran, wheat germ, sprouted millet, parsley, garlic powder, wheatgrass, apples, banana, red or Concord grapes, oranges and sprouted flax.

30. The method of claim 29, wherein a weight of any of the ingredients varies as a percentage of the total weight of the supplement within the range of +1-5%.

31. The method of claim 29, wherein a weight of any of the ingredients varies as a percentage of the total weight of the supplement within the range of one of: +/−10% and +/−50%.

32. A method of equine treatment for reducing physical and mental stress, comprising the step of administering an equine supplement over time, the supplement comprising the following ingredients: sprouted buckwheat, sprouted mung dal, sprouted pumpkin, oat bran, Sensoril®, apples, orange, bananas and honey.

33. The method of claim 32, wherein the honey is raw honey.

34. The method of claim 32, wherein a weight of any of the ingredients varies as a percentage of the total weight of the supplement within the range of +/−5%.

35. The method of claim 32, wherein a weight of any of the ingredients varies as a percentage of the total weight of the supplement within the range of one of: +/−10% and +/−50%.

36. A solid equine-edible matrix having a discrete shaped form for the delivery of nutrients wherein said solid equine-edible matrix provides nutrients suitable for an equine diet that comprises a compressed mixture of at least one grain product including at least one of oat bran and wheat germ and one or more nutrients including honey, said matrix having a maximum moisture content of less than 5% by weight, said matrix having been produced by the process of mixing said grain products with said nutrients; and thereafter removing moisture to a level at or below said maximum moisture content by exposure of said shaped object to a prolonged exposure at a temperature sufficient to achieve the maximum moisture content without destructive effects to said nutrients, whereby moisture is removed from said shaped object resulting in said solid matrix having a discrete shaped form, whereby said solid matrix having a discrete shaped form contains an enhanced level of nutrients.

37. The solid equine-edible matrix of claim 36, wherein said matrix has a maximum moisture content of less than 1% by weight.

38. The solid equine-edible matrix of claim 36, wherein the honey is raw honey.

39. The solid equine-edible matrix of claim 36, wherein the honey is unfiltered honey.
